(19) United States
(12) Patent Application Publication
(10) Pub. No.: US 2025/0339110 A1
(43) Pub. Date: Nov. 6, 2025

(54) METHOD AND APPARATUS FOR SELECTIVE PROJECTIONS FROM DIGITAL TOMOSYNTHESIS

(71) Applicant: CARESTREAM HEALTH, INC., Rochester, NY (US)

(72) Inventors: Luca BOGONI, Philadelphia, PA (US); Levon O. VOGELSANG, Webster, NY (US); William J. SEHNERT, Fairport, NY (US)

(21) Appl. No.: 18/871,172

(22) PCT Filed: Jun. 28, 2023
(86) PCT No.: PCT/US2023/069252

§ 371 Date: Dec. 3, 2024

Related U.S. Application Data
(60) Provisional application No. 63/357,028, filed on Jun. 30, 2022.

(51) Int. Cl. A61B 6/02; A61B 6/00; A61B 6/46

(52) U.S. Cl. CPC A61B 6/025; A61B 6/469; A61B 6/5223

(57) ABSTRACT

Selective projections from a digital tomosynthesis image acquisition may be used to select one or more regions of interest therein, and identify regions that are not of interest and which may be excluded. The non selected regions may be deleted from the digital tomosynthesis image acquisition and only the selected regions of interest within the digital tomosynthesis image acquisition are rendered and projected.

### BACKGROUND OF THE INVENTION

The subject matter disclosed herein relates to digital tomosynthesis. In particular, to an apparatus and method of selectively deleting obstructive image data from digital tomosynthesis projections.

Digital tomosynthesis (DT) is a process that acquires radiographic images using a stationary digital radiographic (DR) detector and a mobile x-ray source, or multiple selectively arranged stationary x-ray sources, which radiates x-rays from a selected number of positions, as illustrated in FIG. 1A. A reconstruction process then triangulates the acquired DT radiographic image data to generate a 3D reconstruction for the imaged object, as shown in FIG. 1B, and which enables projecting the acquired DT image information into various 2D images (slices) as desired.

As illustrated in FIGS. 2A-2B, the acquisition and reconstruction process may be used to generate a series of images reconstructed on a plane orthogonal to the middle position of the x-ray source, i.e., position s2 in FIG. 1B. FIG. 2A is an exemplary radiographic image showing a conventional chest x-ray AP acquisition. FIG. 2B is a lateral acquisition of a chest x-ray with an overlay illustrating the coronal planes 201, which are perpendicular to the page, as shown, and represented by dashed vertical lines, of the DT reconstructions.

Digital tomosynthesis offers the advantage of undoing some of the effects of superimposing anatomical structures that are a byproduct of a normal x-ray projection image acquisition, as shown in FIG. 1A. This provides better anatomical discrimination and a higher diagnostic ability and confidence. Radiographic techniques have been established to image the body from specific angles/poses/views to highlight an anatomy of interest in x-ray imaging. Radiographers undergo substantial training to be able to align patients to achieve maximum visibility of certain body regions so as to increase diagnostic discriminability. These specialized views may still include the full anatomy in the field of view of the projection which may be obstructing and impacting the clinical interpretation. Furthermore, images with optimal image content depend on the expertise and experience of the radiographer to align the patient according to the correct plane and the patient's ability to comply with the posture for the duration of the image acquisition procedure. Often, these acquisition procedures are subject to retakes thus exposing the patient to additional ionizing radiation.

In spite of providing the ability of separating projected anatomical structures into a multitude of projections, and offering essentially a 3D image, DT has not been typically perceived as a modality of choice. While it has been recognized to offer clinical utility, there is an inherent limitation on how the image information is presented which is hindering its adoption. DT introduces a new workflow for a radiologist who is normally used to reviewing a single x-ray image; a significant increase the amount of time required to review the case; and requires new interpretation skills to visualize the patient anatomy in 3D. These, as well as other considerations, such as increase in data storage requirements due to the large number of radiographic images acquired when compared to an individual x-ray, may hamper the adoption of this modality and thwart the clinical benefit that can be derived.

Slabbing is a well-known alternative visual presentation mechanism for reducing reviewing-time of radiographic images of patients while maintaining anatomical discriminability. This capability is available as a standard feature on many imaging systems. When reviewing a computed tomography (CT) dataset, which might include upward of 500 images for a CT image set when reconstructed at 1 mm slice thickness, a radiologist might slab, or combine, selected ones of the slices into a 5 mm or a 10 mm slab using a variety of algorithms such as maximum intensity projection (MIP). In this manner the number of slices to review are reduced while maintaining anatomical discrimination. Slabbing is routinely used clinically to perform a fast-case review, while retaining the availability of the original thin slices which may be used for disambiguation purposes.

FIGS. 3A-3C illustrate the creation of two exemplary slabs that emphasize medial and posterior anatomy. Slabs include image slices that are typically grouped, added, or stacked, using a formula irrespective of the anatomy they contain. For example, FIG. 3B shows a slab-view of three selected coronal slices 301, indicated at the top of FIG. 3A, combined, or added, to generate the slab of FIG. 3B which highlights the medial respiratory area. FIG. 3C shows a slab-view of three different selected coronal slices 302, indicated at the bottom of FIG. 3A, combined, or added, to generate the slab of FIG. 3C which highlights the posterior spine and ribs.

The discussion above is merely provided for general background information and is not intended to be used as an aid in determining the scope of the claimed subject matter.

### BRIEF DESCRIPTION OF THE INVENTION

In one embodiment, the present disclosure proposes a methodology for the creation of selective projections from digital tomosynthesis reconstructions by: (1) identifying anatomical regions of interest, or structures, within the 3D volume (such as in a sequence of slices); (2) projecting selected ones of these regions of interest from a selected number of slides to achieve (3) a projection that provides a clearer visualization of the selected region of interest. This method does not introduce any transformations to the original reconstructed slides, rather, it enhances the visualization by removing encumbering image information.

In one embodiment, a portion of a patient body may be segmented coarsely and qualitatively, such as in anterior surface, anterior, medial (possibly refined as left, center, right), posterior, and posterior surface. This type of qualitative/semantic segmentation may be used to produce five selected stacked projections, each emphasizing one of the segmented regions. In the example of FIGS. 3A-3C described herein, two selected stacked projections are described. In the context of digital chest tomosynthesis (DCT) the method and apparatus described herein may be used to enhance the stacked images in order to selectively capture (i) the anterior surface image data which may include all the external tubing, lines and apparatuses outside the patient body which image data may be selected to be ignored, or excluded, but may be used for completeness and verification purposes; (ii) the anterior projection image data which may include the anterior rib-cage and sternum; (iii) the medial left and right image data having the lung areas, whereas the medial central image data may capture the airways (trachea), aorta, vena-cava and heart; (iv) the posterior image data would capture the back ribs and spine; and/or (v) the posterior surface image data may be used as the anterior surface to provide a demarcation to separate and exclude, or discard, any visual effect resulting from such background image data.

As a byproduct of this process, the radiologist, in addition to the x-ray reconstructed from a DT acquisition, or a DT volume, may have these anatomically selective stacked projections to visualize at a glance the entire DT volume. This effectively enables creation of slabs which contain all of the desired anatomy without being encumbered by image data from other anatomy or devices that are not desired to be included.

Selective projections from a digital tomosynthesis image acquisition may be used to select one or more regions of interest therein, and identify regions that are not of interest and which may be excluded, or deleted. The non-selected regions may be deleted from the digital tomosynthesis image acquisition and only the selected regions of interest within the digital tomosynthesis image acquisition are rendered and projected.

In one embodiment, a method of generating selective projections from a digital tomosynthesis acquisition selects one or more regions of interest within the digital tomosynthesis image acquisition, then identifies regions from the digital tomosynthesis image acquisition that are not in the selected one or more regions of interest and deletes image data of the identified regions. Image data only from the selected one or more regions of interest is rendered and projected.

In one embodiment, a method of generating selective projections from a digital tomosynthesis image acquisition selects one or more regions of interest within the digital tomosynthesis image acquisition, and projects image data only from the selected one or more regions of interest within the digital tomosynthesis image acquisition.

The summary descriptions above are not meant to describe individual separate embodiments whose elements are not interchangeable. In fact, many of the elements described as related to'a particular embodiment can be used together with, and possibly interchanged with, elements of other described embodiments. Many changes and modifications may be made within the scope of the present invention without departing from the spirit thereof, and the invention includes all such modifications.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings below are intended to be drawn neither to any precise scale with respect to relative size, angular relationship, relative position, or timing relationship, nor to any combinational relationship with respect to interchangeability, substitution, or representation of a required implementation., emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIGS. 1A-1B are schematic diagrams showing tomosynthesis process image acquisition (FIG. 1A), and reconstruction (FIG. 1B);

FIGS. 2A-2B are radiographic images showing a conventional chest x-ray AP acquisition (FIG. 2A) and a lateral acquisition of chest x-ray (FIG. 2B) with an overlay illustrating the coronal planes of DT reconstructions;

FIGS. 3A-3C show a visualization of two image sets (slabs) each having three coronal slices (FIG. 3A) combined to generate a slab having a medial portion highlighting the respiratory area (FIG. 3B) and the posterior portion highlighting spine and ribs (FIG. 3C);

FIG. 4 shows an actual lateral chest x-ray, where the vertical dashed lines illustrate the coronal image slices that include the spine while the vertical boxes illustrate portions of the image data that may be excluded from the generation of the slab;

FIG. 5 shows a visualization of regions 1, 2, 3 and a heart region 4 overlaid on a DT projection image;

FIG. 6 shows an anatomical region region of interest representing the spinal column, which is expanded to include the anterior and posterior portion of the vertebral column; and

FIG. 7 shows an exemplary mask used for segmenting an anatomical region of interest.

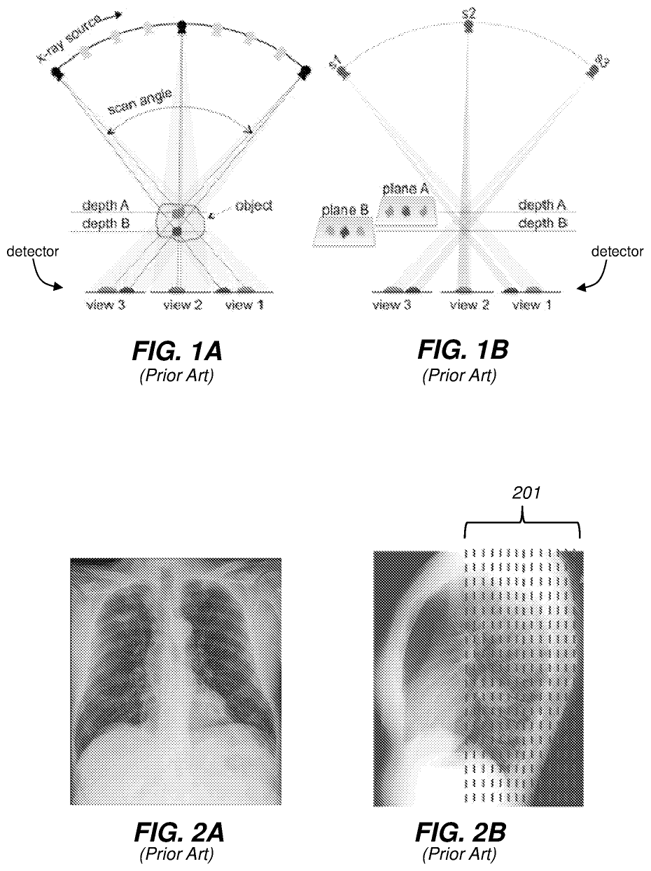

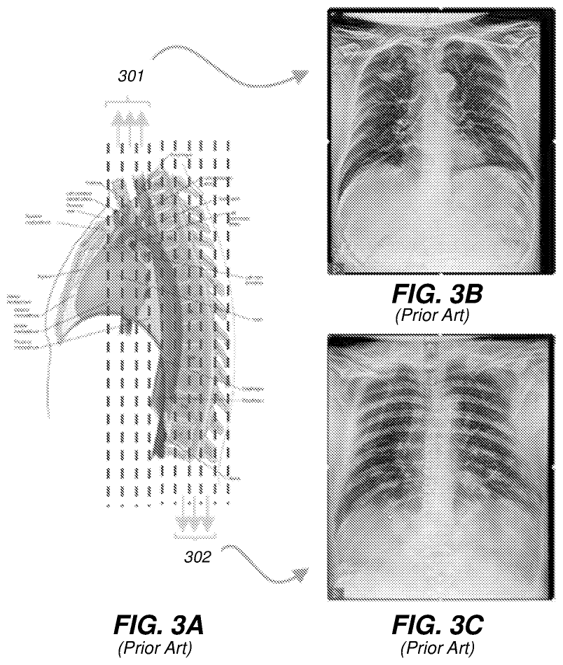

### DETAILED DESCRIPTION OF THE INVENTION

This application claims priority to U.S. Patent Application Ser. No. 63/357,028, filed Jun. 30, 2022, in the name of Bogoni et al., and entitled ANATOMICALLY SELECTIVE PROJECTIONS FROM DIGITAL TOMOSYNTHESIS, which is hereby incorporated by reference herein in its entirety.

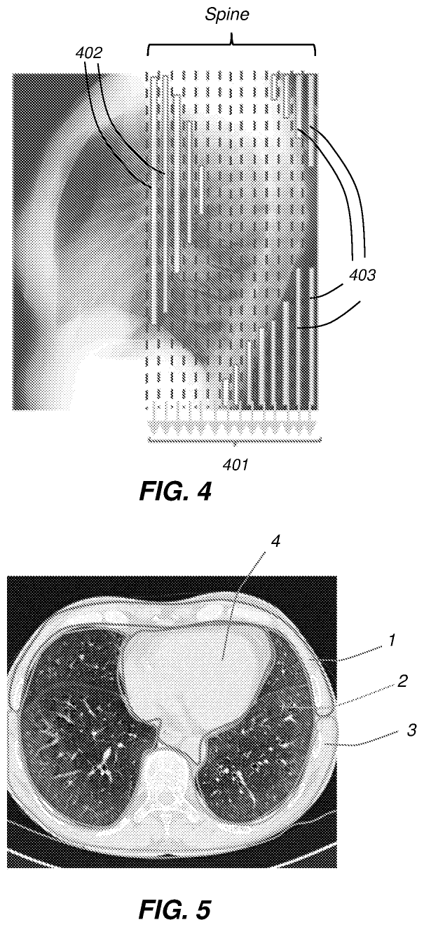

FIG. 4 shows an actual lateral chest x-ray image. Increasing the number of slices included in a slab, dramatically illustrated by the dashed vertical lines 401 in FIG. 4, may capture all of the desired posterior skeletal anatomy—back spine, posterior ribs and spinal processes, however, this slab also includes significant obstructing data, represented by the elongated vertical boxes 402, 403, of FIG. 4. These elongated vertical boxes include radiographic image data containing undesired medial anatomy represented by the vertical boxes 402, and image data outside of the patient's body represented by the vertical boxes 403. While this rendered view would provide more anatomical discrimination than a single x-ray of the chest, it would greatly hamper the benefits that may be derived from digital tomosynthesis. One feature of the presently described invention addresses the elongated vertical boxes 402, 403, in FIG. 4 which illustrate portions of the DT image data that may be deleted, or excluded, from the generation of the slab.

When anatomical structures are removed due to excluding selected regions of the projections, the radiologist may lose context in viewing image slabs formed from the remaining areas of image data. For instance, when a radiologist views disease manifestations in the slabs, such as COPD in the airways or the presence of lung nodules, the selective removal, or exclusion, of image data regions in the slices that show the posterior ribs or indwelling devices may make it difficult for the physician to include a location reference in a report. Therefore, it might be convenient to include in a particular slab one or more projection regions previously selected for exclusion in order to provide a location reference. In one embodiment, these selected projection regions may be re-included but modified to have a user selected level of transparency in order to display, in the slab, an anatomical structure or object that may be used as a visual reference. In one embodiment, a selected anatomical projection may be augmented by processing the image data of the anatomical reference to enhance the referential structure, such as by adding color, shading, or a selected different level of transparency in order to highlight it.

In another embodiment, anatomical areas in digital tomosynthesis acquisitions may be identified as part of a segmentation or clustering process. When generating a selective projection of the anatomical areas a mask may applied to the DT images, so that only areas of image data within the mask are selected to generate a projection, while areas of image data outside the mask are excluded, or deleted. In another embodiment, unwanted slices that display regions external to the patient body or on its surface may be omitted from a slab to visualize only images which contain anatomy, and do not contain objects or structures external to the body of the patient being imaged. In another embodiment, a projection of the rib cage as well as the spine may be projected using a cylindrical projection centered around the heart thus yielding an unfolded view of the ribs. This projection would offer a better view of the ribs and possibly support easier fracture detection. In another embodiment, the projection may be generated for selective areas identified as a byproduct of using an automatic tool for the identification. In this instance the ability to identify the regions may offer means of separating them from the surrounding anatomy and at the same enhancing them to gain more visibility and diagnostic discrimination.

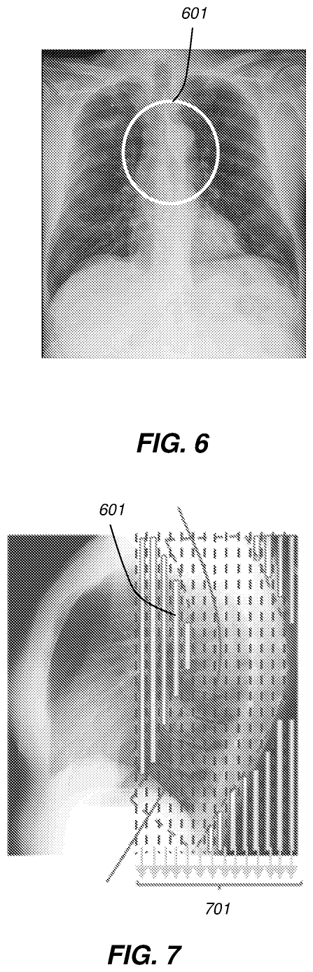

FIG. 5 illustrates the embodiment wherein anatomical areas in digital tomosynthesis acquisitions may be identified as part of a segmentation or clustering process. In this embodiment, the regions to be enhanced by selective projection may be manually identified or specified by user selection via a user interface. Oblique views collected using DT allows clearer visualization of the anatomy of interest. As shown in FIG. 5, segmented regions 1-4 are shown, as drawn or selected by a user via a user interface. In this example, segmented region of interest 3 may be selected by the user for enhanced visualization. FIG. 6 illustrates another example of a user interface whereby a region to be excluded 601, or deleted, may be selected via a user interface.

FIG. 7 illustrates the resulting slab generated by combining the DT slices 701. The portions of slices 701 that do not contribute to the image enhancement of the region of interest 3 of FIG. 5, will be excluded, or deleted, from the combination of slices 701. These excluded portions are represented by the elongated boxes illustrated in FIG. 7, and correspond to the elongated boxes 402, 403, as described herein in relation to FIG. 4. The excluded portion 601, selected via a user interface as in the example of FIG. 6, is also indicated in FIG. 7.

In another embodiment, specialized views may have associated mechanisms, such as neural nets, designed to generate a segmentation of the anatomical structure(s) associated with the specific view. In each of the embodiments discussed herein, the area of interest, may be augmented following segmentation, so as to provide context as well as a buffer area to make sure that anatomical areas of interest have not been under-segmented. As described herein, the segmented structures may include indwelling devices, so as to provide a clear view of the devices and any possible damages or improper localization which might have occurred. Ability of visualizing these may lead to timely clinical interventions/interactions.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language. These computer program instructions may be provided to a processor of a general purpose computer, special purpose computer, or other programmable data processing apparatus to produce a machine, such that the instructions, which execute via the processor of the computer or other programmable data processing apparatus. These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method of generating selective projections from a digital tomosynthesis (DT) image acquisition, the method comprising:
selecting one or more regions of interest within the digital tomosynthesis image acquisition;
identifying regions from the digital tomosynthesis image acquisition that are not in the selected one or more regions of interest and deleting image data of the identified regions from the digital tomosynthesis image acquisition; and
rendering and projecting image data only from the selected one or more regions of interest within the digital tomosynthesis image acquisition.

2. The method of claim 1, further comprising selecting the one or more regions of interest based on a specific selected anatomy included in the digital tomosynthesis image acquisition.

3. The method of claim 2, further comprising activating an automatic algorithm to identify the selected one or more regions of interest.

4. The method of claim 3, further comprising an operator manipulating a user interface to interactively select, outline, or modify the selected one or more regions of interest.

5. The method of claim 1, wherein the step of projecting includes projecting on one of a planar or a non-planar surface.

6. The method of claim 1, wherein the step of projecting includes projecting on a curvilinear surface.

7. A method of generating selective projections from a digital tomosynthesis (DT) image acquisition, the method comprising:
selecting one or more regions of interest within the digital tomosynthesis image acquisition; and
rendering and projecting image data only from the selected one or more regions of interest within the digital tomosynthesis image acquisition.

8. The method of claim 7, further comprising identifying regions in the digital tomosynthesis image acquisition to be deleted from the digital tomosynthesis image acquisition.

9. The method of claim 8, further comprising selecting the one or more regions of interest based on a specific selected anatomy included in the digital tomosynthesis image acquisition.

10. The method of claim 8, further comprising activating an automatic algorithm to identify the selected one or more regions of interest.

11. The method of claim 9, further comprising an operator manipulating a user interface to interactively select, outline, or modify the selected one or more regions of interest.

12. The method of claim 7, wherein the step of projecting includes projecting on one of a planar or a non-planar surface.

13. The method of claim 7, wherein the step of projecting includes projecting on a curvilinear surface.
